(19) United States
(12) Patent Application Publication
(10) Pub. No.: US 2026/0076698 A1
(43) Pub. Date: Mar. 19, 2026

(54) ABLATION MEDICAL DEVICE WITH THE IMAGING CAPABILITIES

(71) Applicant: Boston Scientific Medical Device Limited, Galway (IE)

(72) Inventor: Meghana Akella, Hyderabad (IN)

(73) Assignee: Boston Scientific Medical Device Limited, Galway (IE)

(21) Appl. No.: 19/332,683

(22) Filed: Sep. 18, 2025

Related U.S. Application Data
(60) Provisional application No. 63/696,138, filed on Sep. 18, 2024.

(51) Int. Cl. A61B 17/22; A61B 17/00; A61B 90/00

(52) U.S. Cl. CPC A61B 17/2202; A61B 17/00234; A61B 90/37; A61B 90/39; A61B 2017/00137; A61B 2017/00238; A61B 2017/00292; A61B 2017/22028; A61B 2090/3784

(57) ABSTRACT

Medical device systems and methods for using medical devices/systems are disclosed. An example medical device system may include a system for treating a calcified blood vessel. The system may include an elongate catheter shaft having a distal end region. A plurality of imaging transducers may be disposed along the distal end region of the elongate catheter shaft. A plurality of ablation transducers may be disposed along the distal end region of the elongate catheter shaft and positioned adjacent to the plurality of imaging transducers. The plurality of ablation transducers may include ablation transducers spaced circumferentially around the elongate catheter shaft, ablation transducers axially-spaced along the elongate catheter shaft, or both ablation transducers spaced circumferentially around the elongate catheter shaft and ablation transducers axially-spaced along the elongate catheter shaft.

### CROSS-REFERENCE TO RELATED APPLICATIONS

This application claims the benefit of priority under 35 U.S.C. § 119 of U.S. Provisional Application No. 63/696,138, filed Sep. 18, 2024, the entire disclosure of which is hereby incorporated by reference.

### TECHNICAL FIELD

The present disclosure pertains to medical devices and methods for manufacturing medical devices. More particularly, the present disclosure pertains to ablation medical devices with imaging capabilities.

### BACKGROUND

A wide variety of medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A system for treating a calcified blood vessel is disclosed. The system comprises: an elongate catheter shaft having a distal end region; a plurality of imaging transducers disposed along the distal end region of the elongate catheter shaft; and a plurality of ablation transducers disposed along the distal end region of the elongate catheter shaft and positioned adjacent to the plurality of imaging transducers; wherein the plurality of ablation transducers includes ablation transducers spaced circumferentially around the elongate catheter shaft, ablation transducers axially-spaced along the elongate catheter shaft, or both ablation transducers spaced circumferentially around the elongate catheter shaft and ablation transducers axially-spaced along the elongate catheter shaft.

Alternatively or additionally to any of the embodiments above, at least some of the plurality of ablation transducers are arranged in an array that has an arcuate shape.

Alternatively or additionally to any of the embodiments above, at least some of the plurality of ablation transducers are arranged in an array that has a planar shape.

Alternatively or additionally to any of the embodiments above, the distal end region of the elongate catheter shaft includes a cutout region and wherein at least some of the plurality of ablation transducers are disposed within the cutout region.

Alternatively or additionally to any of the embodiments above, at least some of the plurality of ablation transducers include dual mode transducers.

Alternatively or additionally to any of the embodiments above, at least some of the plurality of ablation transducers include ultrasound transducers.

Alternatively or additionally to any of the embodiments above, at least some of the plurality of imaging transducers include ultrasound transducers.

Alternatively or additionally to any of the embodiments above, at least some of the plurality of ablation transducers are capacitive micromachined ultrasound transducers.

Alternatively or additionally to any of the embodiments above, at least some of the plurality of ablation transducers are dual mode capacitive micromachined ultrasound transducers capable of both an imaging modality and an ablation modality.

A system for treating a calcified blood vessel is disclosed. The system comprises: an elongate catheter shaft having a distal end region; a plurality of imaging transducers disposed along the distal end region of the elongate catheter shaft; a plurality of ablation transducers disposed along the distal end region of the elongate catheter shaft and positioned adjacent to the plurality of imaging transducers; wherein at least some of the plurality of ablation transducers are capacitive micromachined ultrasound transducers; and wherein the plurality of ablation transducers includes a plurality of circumferential arrays of ablation transducers spaced circumferentially around the elongate catheter shaft, and/or a plurality of axially-spaced arrays of ablation transducers axially-spaced along the elongate catheter shaft.

Alternatively or additionally to any of the embodiments above, further comprising a plurality of linear fiducial markings disposed between the plurality of imaging transducers and the plurality of ablation transducers.

Alternatively or additionally to any of the embodiments above, the plurality of linear fiducial markings disposed partially along the distal end region of the elongate catheter shaft and circumferentially around the elongate catheter shaft.]

Alternatively or additionally to any of the embodiments above, at least some of the plurality of ablation transducers are dual mode transducers.

Alternatively or additionally to any of the embodiments above, wherein at least some of the plurality of ablation transducers are dual mode capacitive micromachined ultrasound transducers capable of both an imaging modality and an ablation modality.

Alternatively or additionally to any of the embodiments above, at least some of the plurality of imaging transducers are capacitive micromachined ultrasound transducers.

Alternatively or additionally to any of the embodiments above, the plurality of ablation transducers operate at power densities ranging from 50 to 80 W/cm².

Alternatively or additionally to any of the embodiments above, wherein the plurality of ablation transducers operate at frequencies ranging 20 Hz to 60 MHz.

Alternatively or additionally to any of the embodiments above, at least some of the circumferential arrays of ablation transducers, at least some of the axially-spaced arrays of ablation transducers, or both have an arcuate shape.

Alternatively or additionally to any of the embodiments above, at least some of the circumferential arrays of ablation transducers, at least some of the axially-spaced arrays of ablation transducers, or both have a planar shape.

A method for treating a calcified blood vessel is disclosed. The method comprises: advancing a catheter system to a treatment site, the catheter system comprising: an elongate catheter shaft having a distal end region; a plurality of imaging transducers disposed along the distal end region of the elongate catheter shaft; a plurality of ablation transducers disposed along the distal end region of the elongate catheter shaft and positioned adjacent to the plurality of imaging transducers; wherein the plurality of ablation transducers includes ablation transducers spaced circumferentially around the elongate catheter shaft, ablation transducers axially-spaced along the elongate catheter shaft, or both ablation transducers spaced circumferentially around the elongate catheter shaft and ablation transducers axially-spaced along the elongate catheter shaft; and activating at least some of the plurality of imaging transducers and/or some of the plurality of ablation transducers.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view depicting an example medical device disposed in a blood vessel.

FIG. 2 is a partially cutaway portion of an example medical device.

FIG. 3 is a partially cutaway portion of an example medical device.

FIG. 4 is a side view of a distal portion of an example medical device.

FIG. 5 is a top view of a distal portion of an example medical device.

FIG. 6 is a cross-sectional view of a portion of an example medical device.

FIG. 7 is a cross-sectional view of a portion of an example medical device.

FIG. 8 is a flow chart depicting a method for treating a calcified blood vessel using an example medical device.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

A number of different clinical interventions are known that can be used to treat peripheral arteries including arteries below-the-knee. Traditional approaches such as angioplasty and stenting are well-established, offering immediate relief by restoring blood flow through mechanical means. Angioplasty involves the insertion of a catheter with a deflated balloon into the narrowed artery, which is then inflated to compress the plaque against the artery walls, widening the vessel and improving blood flow. Stenting, another established technique, involves placing a small metal mesh tube (stent) into the artery post-angioplasty to keep it open and prevent re-narrowing (restenosis). Stents may be bare metal or drug-eluting, the latter releasing medication over time to inhibit tissue growth that could lead to re-narrowing. In recent years, advancements in endovascular techniques, including atherectomy and drug-coated balloons, have provided additional options for managing complex lesions and improving long-term outcomes.

Occasionally, peripheral arteries in need of treatment can be calcified, which may complicate treatment. Calcified arteries are hardened due to the deposition of calcium within the arterial walls. This calcification makes the arteries less flexible and more resistant to traditional treatments like angioplasty and stenting. The hardened plaque can be difficult to compress with a balloon during angioplasty, limiting the effectiveness of this approach in widening the artery. Calcified arteries often have a poorer long-term prognosis compared to non-calcified arteries. They are more prone to recurrent narrowing (restenosis) and may require repeat interventions or more complex treatments over time to maintain adequate blood flow. This contributes to increased healthcare costs and patient morbidity. The technical difficulties associated with navigating through and treating calcified lesions can prolong procedure times and increase radiation exposure to both patients and medical staff. This highlights the need for innovative solutions that can effectively manage calcified lesions while minimizing procedural risks.

Disclosed herein are catheter systems generally designed for the visualization and/or treatment of arteries, for example calcified arteries below-the-knee. This capability not only enhances the efficacy of treatments but also reduces procedural risks by minimizing the potential for arterial dissection and ensuring more durable outcomes. Some additional details regarding these catheter systems can be found herein. Furthermore, the system includes provisions for post-operative therapy, where it can be used externally to manage early calcium re-growth, thereby extending the effectiveness of initial interventions. By combining therapeutic precision with comprehensive imaging capabilities, this novel catheter system represents a significant advancement in the management of calcified peripheral arteries, offering improved patient outcomes and reduced healthcare burdens.

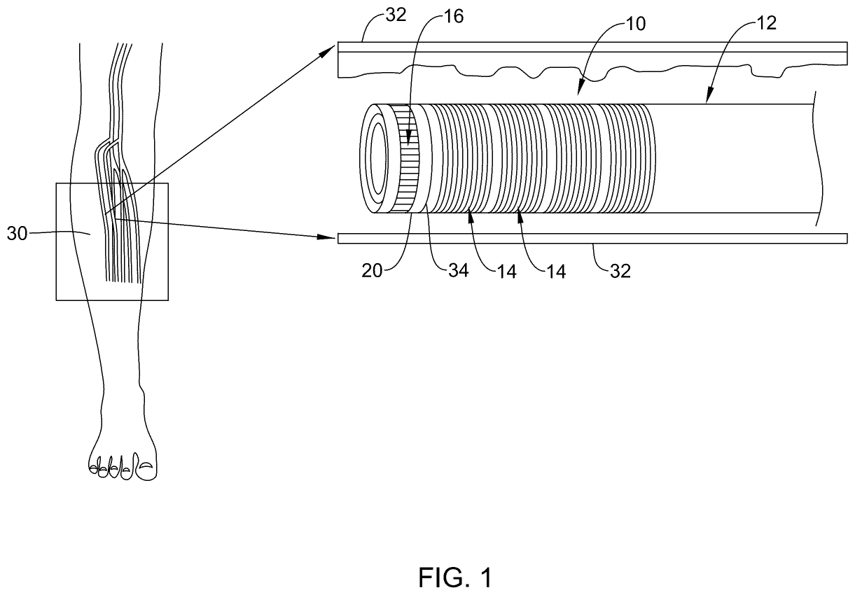

FIG. 1 schematically depicts an example catheter system 10 disposed in a blood vessel 32 of a patient. In this example, the blood vessel 32 is a peripheral blood vessel below-the-knee, and the system is used for treating a calcified blood vessel (e.g., a calcified artery below a knee). In some instances, the system 10 may be navigated to a target region 30, for example along a patient's leg (e.g., below-the-knee) where the system 10 may be used to treat the blood vessel 32. While the system is capable of targeting areas below-the-knee, treating calcified blood vessels, or blood vessels in general, in a variety of locations is contemplated. For example, the system may be used to treat arteries, veins, peripheral vessels, cardiac vessels, and/or the like.

In some instances, the system 10 includes an elongated catheter shaft 12 with a distal end region 20. A plurality of transducers may be disposed along the distal end region 20 of the elongate catheter shaft 12. For example, one or more ablation transducers or ablation transducer arrays 14 may be disposed along the distal end region 20 of the elongate catheter shaft 12. In some of these and in other instances, one or more imaging transducers or imaging transducer arrays 16 may be disposed along the distal end region 20 of the elongate catheter shaft 12. In some instances, the ablation transducer arrays 14 may be disposed adjacent to the imaging transducer arrays 16.

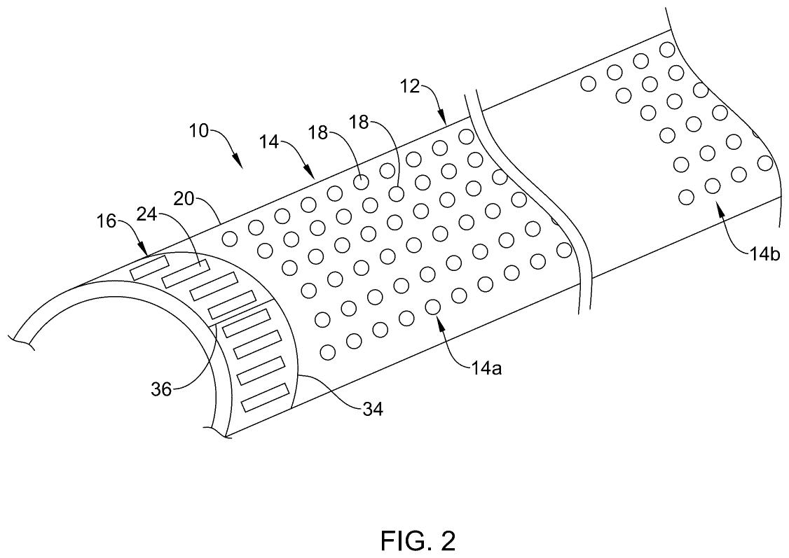

The ablation transducer arrays 14 may include a series of individual ablation transducer elements 18 as shown in FIG. 2. The ablation transducer arrays 14 may be arranged/configured on or around the catheter shaft 12 as a plurality of rings, for example rings 14a, 14b, formed by the individual transducer elements 18. FIG. 1 shows four rings of ablation transducer arrays 14. This is not intended to be limiting as other numbers are contemplated including 1, 2, 3, 4, 5, 6, 7, 8, 9. 10, or more rings. The ring configuration and/or arcuate shape may allow for uniform energy delivery around the circumference of the catheter shaft 12, ensuring that the entire targeted area receives consistent treatment. The ablation transducer elements 18 may be spaced circumferentially around the elongate catheter shaft 12, spaced axially along the catheter shaft 12, or spaced both circumferentially and axially along the elongate catheter shaft 12. These arrangements may provide a linear or segmented ablation capability, allowing the physician to treat different sections of the artery sequentially or simultaneously, depending on the clinical requirements. The axial spacing can be adjusted to control the overlap or gaps between the treated zones, ensuring precise coverage of the calcified area.

Similarly, the imaging transducer arrays 16 may include a series of individual imaging transducer elements 24. In some instances, the imaging transducer arrays 16 may be disposed along the distal end region 20 of the elongate catheter shaft 12 and may be arranged in a ring or array that has an arcuate shape. The imaging transducer elements 24 may be spaced circumferentially around the elongate catheter shaft 12, spaced axially along the catheter shaft 12, or spaced both circumferentially and axially along the elongate catheter shaft 12.

In addition to rings, the ablation transducer elements 18 and/or the imaging transducer elements 24 may be arranged in various configurations to optimize their function. A radial configuration involves placing the transducers around the shaft 12 in a pattern that points either outward or inward, while a helical configuration arranges them in a spiral pattern along the shaft's length. Another option is a concentric rings configuration, where transducers are placed in multiple rings around the elongated catheter shaft. Alternatively, a random distribution can be used, scattering transducers irregularly across the surface. These configurations can also be combined, such as by integrating concentric rings with radial arrays or combining helical patterns with other configurations, depending on the specific application and desired outcomes.

In some instances, the imaging transducer arrays 16 and/or ablation transducer arrays 14 (and/or the individual transducer elements 18, 24), may include ultrasound transducers. For example, the ablation transducer elements 18 and/or the imaging transducer elements 24 may include high intensity focused ultrasound (HIFU) transducers. In at least some instances, the HIFU transducers may be configured as capacitive micromachined ultrasonic transducers (CMUT) or polymer-based piezoelectric ultrasonic transducers. Such transducers may be desirable for a number of reasons. For example, CMUT/HIFU transducers may be capable of generating a shock wave and/or cavitation that uses acoustofluidic and/or mechanical forces within a blood vessel that may be sufficient to rupture calcium. In addition, CMUT/HIFU transducers may also be configured to be dual mode transducers capable of both ablation and imaging.

In some instances, the innovative device integrates a catheter-based HIFU ablation device with ultrasound imaging capabilities using phased-array transducers. This integration addresses several challenges in current arterial treatment methods. Peripheral arterial disease often presents with calcified arteries, complicating treatment with conventional devices due to reduced arterial compliance and increased risk of stent failure such as under-expansion and malposition. The proposed catheter system introduces a new modality employing mechanical and cavitation forces via HIFU to fracture calcium deposits within arterial walls. This breakthrough not only enhances the efficacy of existing treatments by facilitating easier access and maneuverability of devices within calcified arteries but also addresses post-operative challenges by mitigating early calcium re-growth through targeted external treatment. Moreover, by combining ultrasound imaging and HIFU ablation capabilities in a single catheter, the device streamlines procedural efficiency by eliminating the need to use multiple catheters and/or replace catheters between imaging and treatment phases. This integrated approach promises to advance the field by providing real-time visualization of arterial structures during calcium treatment, optimizing the precision of interventions for both medial and intimal calcifications.

In some instances, the ablation transducer elements 18 and/or the imaging transducer elements 24 may be used as phased array transducers that may produce multiple acoustic focal points capable of treating differing lengths of the blood vessel 32. A focal map may be used and/or programming instructions may be run by a suitable processor to form a focal map. A clinician may use the ablation transducer elements 18 and/or the imaging transducer elements 24, along with the focal map, in order to target particular regions of interest in the blood vessel 32.

The ablation transducers elements 18 may be designed to operate at a specific power density and frequency, optimized for the effective fracture of calcium deposits. A power density in the range of 50 W/cm² to 80 W/cm², coupled with a frequency between 20 Hz and 60 MHz, is crucial for generating the necessary mechanical and acoustofluidic forces to break down calcified plaques. These parameters are carefully selected to delivery required energy to the calcified regions while minimizing damage to surrounding healthy tissue. The precise control of power density and frequency ensures that the transducers can produce focused ultrasound waves with sufficient intensity to induce micro-fractures within the calcium deposits. This capability is integral to the invention, as it enhances arterial compliance, facilitates easier catheter insertion, and improves overall treatment outcomes. The ability to target and fracture calcified lesions with high precision is what sets this system apart, making it a significant advancement in the treatment of peripheral arterial disease. While the specified power density and frequency range are particularly effective for the intended application, other ranges may also be recommended depending on the specific requirements of different clinical scenarios. The recited specification pertains to one embodiment of the invention and is not intended to limit the scope of the invention.

In some instances, a circumferential fiducial marking 34 may be disposed around the distal end region 20 of the elongate catheter shaft 12 and located between imaging transducer arrays 16 and ablation transducer arrays 14. Additionally, a linear fiducial marking 36 may be disposed along the distal end region 20 of the elongate catheter shaft 12, intersecting with imaging transducer arrays 16 and/or ablation transducer arrays 14. The circumferential fiducial marking 34 and linear fiducial marking 36 both improve positioning and use of the catheter system 10 in a blood vessel 32.

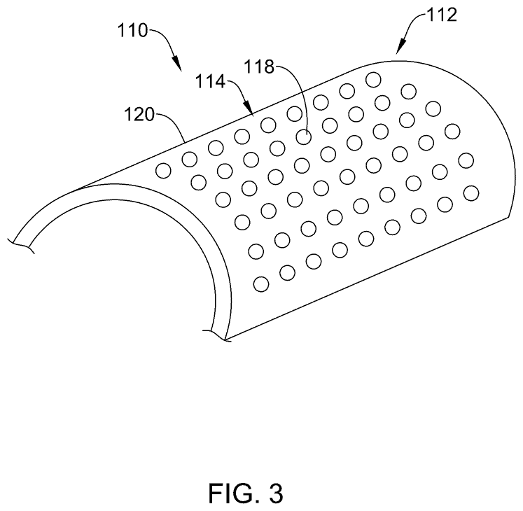

FIG. 3 is a partially cutaway portion of an example medical device system 110, which may be similar in form and function to other systems disclosed herein. The system 110 may include an elongated catheter shaft 112 with a distal end region 120. One or more transducer arrays 114 may be disposed along the distal end region 120 of the elongate catheter shaft 112, including a plurality of individual transducers 118. In this example, the transducers 118 may include micromachined ultrasound transducers capable of both an imaging modality and an ablation modality. In at least some instances, the transducers 118 may include CMUTs, which may have the ability to generate high-frequency ultrasound waves for detailed imaging as well as deliver focused ultrasonic energy for effective ablation. The use of CMUT technology in these dual-mode transducers offers several advantages. For instance, CMUTs can be fabricated with very small dimensions, allowing for a high density of transducer elements on the catheter shaft. This high element density can enhance both the resolution of imaging and the precision of ablation, making it possible to target smaller and more intricate areas of calcification. Additionally, CMUTs are capable of wide bandwidth operation, which is beneficial for producing clear, high-resolution images while also enabling the delivery of controlled, high-intensity energy for ablation. The flexibility of these transducers to operate effectively in both modes makes them a valuable component of the system, contributing to the overall effectiveness and efficiency of the treatment procedure.

These dual-mode transducers are capable of providing real-time imaging to guide the procedure while simultaneously delivering energy for tissue ablation. The transducer array 114 may include a series of particular or individual parts of transducers 118, forming multiple rings along the catheter shaft. The transducer array 114 may be strategically spaced circumferentially around the elongate catheter shaft 112, axially along the shaft, or in a combination of both configurations, depending on the specific requirements of the procedure.

The transducer array 114 may be arranged in various configurations along the distal end region 120, depending on the specific procedural requirements. For example, they can be spaced circumferentially around the elongate catheter shaft 112, providing a 360-degree ablation capability that is particularly effective for treating circular or irregularly shaped calcified deposits. Alternatively, they can be arranged axially along the shaft, allowing for sequential ablation of calcified segments along the length of the artery. In some cases, a combination of both circumferential and axial arrangements is employed to maximize coverage and adaptability, ensuring that the transducer array 114 can be positioned precisely according to the anatomical features of the artery and the extent of the calcification.

By configuring the transducer array 114 as dual-mode transducers for both imaging and ablation, these dual-mode transducers enhance procedural efficiency by eliminating the need for separate imaging and ablation devices and enabling more precise targeting of tissue. This dual functionality facilitates better control during interventions, potentially improving patient outcomes by enabling more accurate ablation guided by immediate imaging feedback.

In some instances, the dual-mode transducers 118 may be configured in a matrix of six by nine, though the number and arrangement can vary depending on the specific application. The configuration shown in the figure is exemplary, and other configurations or arrangements are possible. For instance, in some instances, the transducer array 114 may be arranged in an array with an arcuate shape, which can be advantageous for targeting curved anatomical structures or regions with irregular geometry. In other instances, the transducer array 114 may be arranged in a planar array, which is ideal for treating flat or evenly distributed areas. These various configurations allow for flexibility in the design and application of the system, ensuring that the transducers can be tailored to meet the specific needs of different procedures and anatomical conditions.

In addition to arrays, series, and rings, transducers on the elongated catheter shaft can be arranged in various configurations to optimize their function. A radial configuration involves placing the transducers around the shaft 112 in a pattern that points either outward or inward, while a helical configuration arranges them in a spiral pattern along the shaft's length. Another option is a concentric rings configuration, where transducers are placed in multiple rings around the elongated catheter shaft. Alternatively, a random distribution can be used, scattering transducers irregularly across the surface. These configurations can also be combined, such as by integrating concentric rings with radial arrays or combining helical patterns with other configurations, depending on the specific application and desired outcomes.

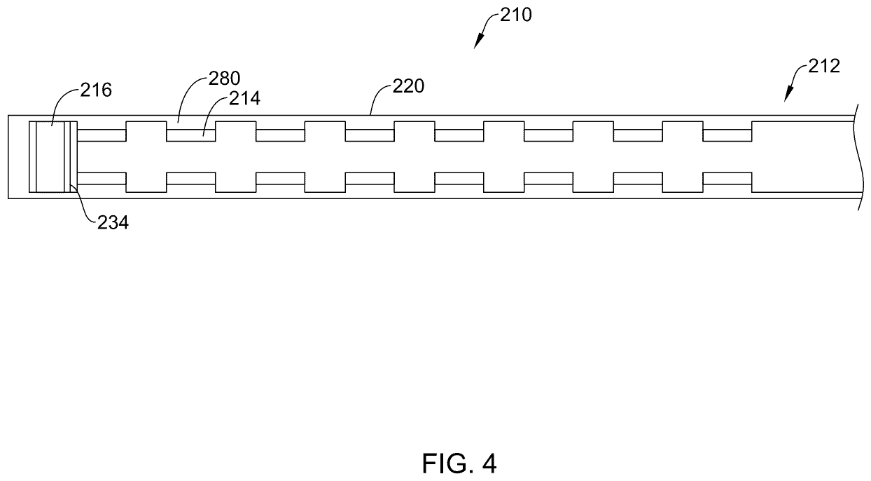

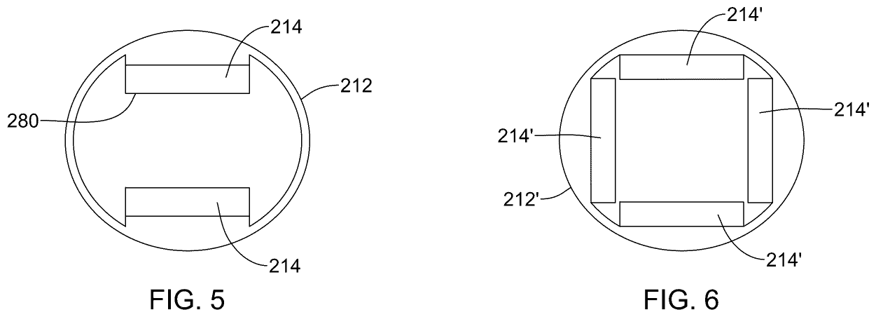

FIGS. 4-5 show a portion of another example medical device system 210 that may be similar in form and function to other systems disclosed herein. The system 210 may include an elongated catheter shaft 212 having a distal end region 220. The distal end region 220 of the elongate catheter shaft 212 may include a plurality of cutout regions 280. Ablation transducer arrays 214 may be disposed in the cutout regions 280. The ablation transducer arrays 214 are positioned adjacent to an imaging transducer array 216 positioned on the distal end region 220 of the catheter shaft 212. The ablation transducer arrays 214 may include a series of individual transducer elements. The ablation transducer arrays 214 can also be configured as a plurality of rings formed by the individual transducer elements. Similarly, the imaging transducer array 216 may include a series of individual imaging transducer elements. In some instances, the cutout regions 280 are arranged circumferentially around the elongate catheter shaft 212, spaced axially along the shaft, or both circumferentially and axially along the elongate catheter shaft 212. FIG. 4 shows sixteen cutout regions and sixteen ablation transducers disposed therein, but both can be other numbers. In some instances, ablation transducer arrays 214 and/or imaging transducer array 216 are arranged in an array that has a planar shape.

The CMUT chips embedded within the catheter body are designed with a flat and square cross-section to achieve sharp focusing during the ablation process. This specific shape enhances the precision of the ultrasound waves, allowing them to be focused more effectively on the calcified areas within the artery. In contrast, a circular cross-section may have difficulty achieving the same level of sharp focus, potentially leading to less effective treatment. The flat and square configuration of the CMUT chips is therefore crucial for maximizing the performance of the catheter system and ensuring optimal outcomes during the procedure.

In some instances, a circumferential fiducial marking 234 is disposed around the distal end region 220 of the elongate catheter shaft 212 and located between imaging transducer array 216 and ablation transducer arrays 214. In some instances, the circumferential fiducial marking 234 is disposed partly around the distal end region 220 of the elongate catheter shaft 212. The circumferential fiducial marking 234 improves positioning and use of the catheter system 210.

In addition to arrays, series, and rings, cutout regions 280 on the elongated catheter shaft can be arranged in various configurations to optimize their function. A radial configuration involves carving out the cutout regions around the shaft 212 in a pattern that points either outward or inward, while a helical configuration arranges them in a spiral pattern along the shaft's length. Another option is a concentric rings configuration, where cutout regions are placed in multiple rings around the elongated catheter shaft. Alternatively, a random distribution can be used, scattering cutout regions irregularly across the surface. These configurations can also be combined, such as by integrating concentric rings with radial arrays or combining helical patterns with other configurations, depending on the specific application and desired outcomes. In some instances, the depth of the cutout regions 280 be within a range of 0.3 mm to 0.7 mm. In other instances, the depth of the cutout regions 280 may be less than 0.3 mm. These variations allow for flexibility in design depending on the desired outcome and operational requirements. The lower the depth of the cutout regions, the more favorable it would be for efficient ultrasound power transmission. Optimally, the depth of the cutout regions should not exceed 1 mm to maintain optimal performance and structural integrity.

In some instances, these ablation transducer arrays 214 may be dual mode capacitive micromachined ultrasound transducers capable of both an imaging modality and an ablation modality. The ablation transducer arrays 214 may include a series of particular or individual parts of transducers, forming multiple rings along the catheter shaft 212.

The arrangement of the transducer arrays 214 may vary. For example, in some instances, the transducer arrays 214 may be embedded into cutout regions 280 of the catheter shaft 212 as shown in FIG. 5. In this example, the cutout regions 280 are arranged on opposite sides of the catheter shaft 212 and a pair of transducer arrays 214 are disposed in the cutout regions 280. Alternatively, a set of transducer arrays 214′ may be disposed about the catheter shaft 212′ as shown in FIG. 6. For example, four transducer arrays 214′ may be disposed about the catheter shaft 212′.

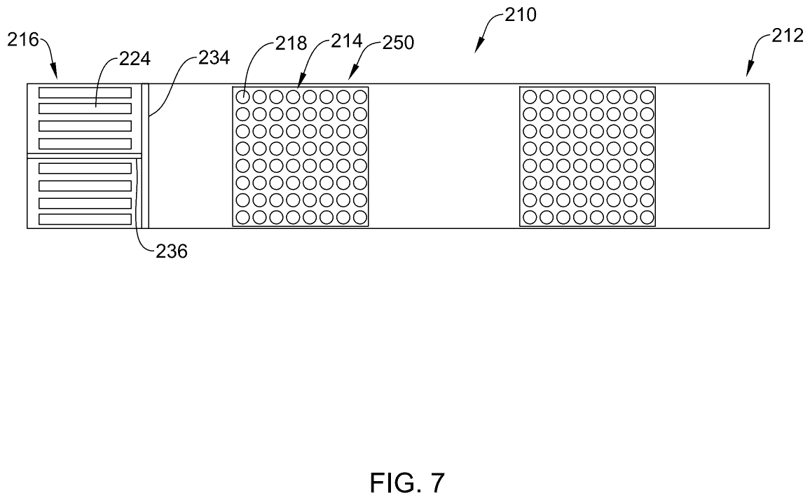

FIG. 7 is a top view of the example medical device system 210. In some instances, the ablation transducer arrays 214 may include a CMUT with an application-specific integrated circuit (ASIC) chip. The ablation transducer arrays 214 may be embedded into the catheter shaft 212 at multiple points. The CMUT may be designed with smaller phased-array HIFU transducers, which may be controlled with a custom software. Each chip may include square or rectangular matrices 250 of ultrasound transducers ranging from rows/columns of 4 to 12. FIG. 7 shows two square matrices of 8 by 8, but both can be other numbers. Keeping the rows and columns smaller ensures enhanced flexibility and steerability. At the distal tip, rectangular CMUT transducers are arranged in arrays ranging from 8 to 64 elements, serving as a series of imaging transducers 216. FIG. 7 shows 8 rows of CMUT transducers, but that can be other numbers. The series of imaging transducers 216 might also include a series of particular or individual parts of imaging transducers 324. The imaging transducer array 216 is distinct from the treatment transducers, such as ablation transducer arrays 214, and may be separated by circumferential fiducial marking 234 and linear fiducial marking 236 utilized for treatment area assessment and parameter calibration. The specific numbers of rows and columns provided are illustrative rather than limiting.

When ASIC chips are combined with CMUTs, the ASIC can be used to control the CMUTs, process the signals they generate, or enhance their performance. This integration allows for more compact, efficient, and precise ultrasound devices, which are particularly useful in medical imaging, therapeutic applications, and non-destructive testing. The ASIC chip can help manage the complex signal processing required for high-resolution imaging or advanced features like real-time data analysis.

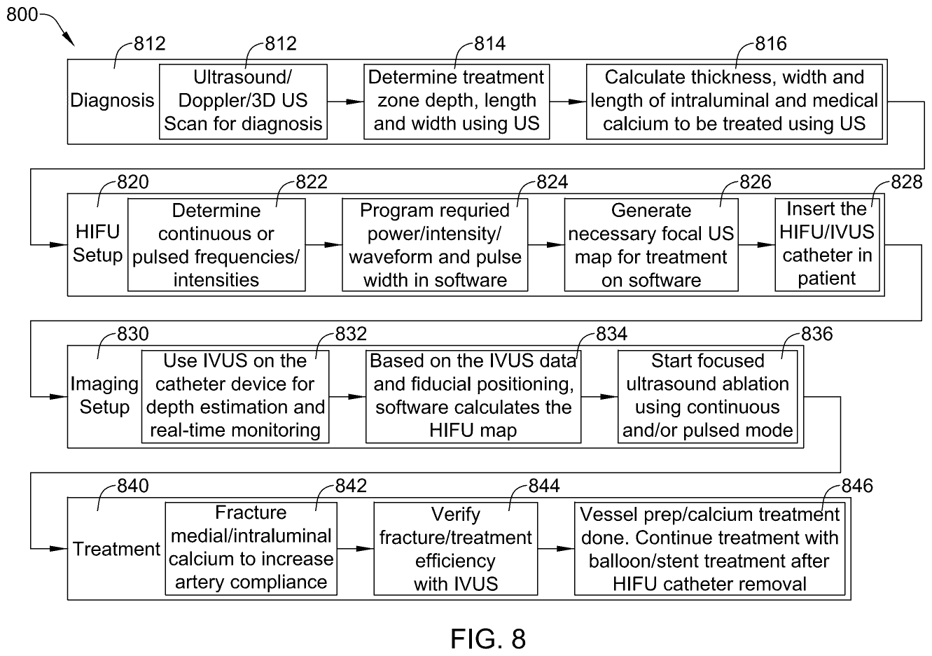

FIG. 8 is a flow chart depicting a method 800 for treating calcified arteries utilizing a catheter-based HIFU ablation approach. The initial diagnostic phase, as indicated at block 810, involves using ultrasound scans to identify medial or intraluminal calcium deposits, as indicated at block 812. This step may allow for accurate identification of the calcified regions that require treatment. In addition to ultrasound, other imaging modalities such as computed tomography (CT) or magnetic resonance imaging (MRI) may be utilized to provide complementary visualization and confirm the presence and extent of the calcifications. After locating the point of interest, depth and thickness profiles of the calcium occlusion may be assessed using ultrasound, 3D ultrasound, or doppler ultrasound, with subsequent measurement of the calcium dimensions. The gathered data allows for precise measurement of the calcium dimensions, including the thickness and extent of the deposits, as indicated at block 814. A clinician may use this data to determine optimal focal ablation points, facilitated by a custom HIFU software generating an acoustic ablation map, as indicated at block 816. The required power for ablation, acoustic patterns and depth of focus required may be determined using the HIFU software.

During HIFU setup, as indicated at block 820, the appropriate mode—either continuous or pulsed—may be selected based on the specific requirements of the treatment, as indicated at block 822. This decision may influence the intensity and duration of the ultrasound waves used for ablation. Once the mode is determined, the catheter may be carefully inserted into the patient, guided by intravascular ultrasound (IVUS) imaging and fiducial markers. These tools may help ensure the catheter is precisely positioned within the treatment zone, directly over the targeted calcified area. The power, frequency, and other wave properties necessary for the ablation are selected based on the recommended guidelines and the specific characteristics of the calcified tissue, as indicated at block 824. The physician then enters these parameters into the HIFU software, which generates the precise ultrasound ablation map tailored to the patient's anatomy, as indicated at block 826. Once the setup is complete and the parameters are verified, the HIFU field is activated, delivering focused ultrasound waves to the targeted calcium deposits, as indicated at block 828. This carefully controlled process ensures that the calcifications are ablated with high precision, minimizing the risk of damage to surrounding healthy tissue and maximizing the effectiveness of the treatment.

The next phase is imaging setup, as indicated at block 830. These tools may help ensure the catheter is precisely positioned within the treatment zone, directly over the targeted calcified area. The IVUS mode may provide real-time, high-resolution imaging of the artery, allowing the physician to clearly visualize the calcium buildup and confirm the catheter's placement relative to the calcifications, as indicated at block 832. This ensures that the treatment is delivered accurately and effectively. Real-time monitoring throughout the procedure is conducted using IVUS, complemented by fiducial markers for alignment of the HIFU setup coordinate system with the treatment region. Calibration verifies the calcium depth from the transducer before initiating the HIFU field. IVUS continues to monitor treatment efficacy, signaling completion upon achieving the desired arterial compliance. Furthermore, software can generate a HIFU map based on data from IVUS and fiducial positioning, as indicated at block 834. External ultrasound or virtual ultrasound (VUS) may provide additional real-time imaging during subsequent treatment stages. Then a focused ultrasound ablation using continuous and/or pulsed mode is initiated, as indicated at block 836.

During the treatment phase, as indicated at block 840, the HIFU is utilized to fracture medial or intraluminal calcium to increase artery compliance, as indicated at block 842. The effectiveness of this calcium fracture is continually verified through IVUS imaging, which ensures that the desired therapeutic outcomes are being achieved, as indicated at block 844. Once the calcium deposits have been sufficiently fractured and the arterial compliance improved, the HIFU/IVUS catheter is carefully withdrawn from the artery. At this point, the procedure advances to the introduction of additional treatment devices such as stents, balloons, or drug-eluting stents (DES), as indicated at block 846. These devices are deployed at the site of treatment to finalize the therapeutic process, ensuring that the artery is appropriately supported, and the overall treatment is completed successfully.

The materials that can be used for the various components of the system 10 (and/or other systems disclosed herein) may include those commonly associated with medical devices. For simplicity purposes, the following discussion makes reference to the shaft 12 and other components of the system 10. However, this is not intended to limit the devices and methods described herein, as the discussion may be applied to other similar tubular members and/or components of tubular members or devices disclosed herein.

The shaft 12, 112, 212, 312 and/or other components of the system 10, 110, 210, 310 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-20 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some instances the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N12076 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N12665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some instances, portions or all of the system 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system 10 to achieve the same result.

In some instances, a degree of Magnetic Resonance Imaging (MRI) compatibility is imparted into the system 10. For example, the system 10, or portions thereof, may be made of a material that does not substantially distort the image and create substantial artifacts (e.g., gaps in the image). Certain ferromagnetic materials, for example, may not be suitable because they may create artifacts in an MRI image. The system 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A system for treating a calcified blood vessel, the system comprising:
an elongate catheter shaft having a distal end region;
a plurality of imaging transducers disposed along the distal end region of the elongate catheter shaft; and
a plurality of ablation transducers disposed along the distal end region of the elongate catheter shaft and positioned adjacent to the plurality of imaging transducers;
wherein the plurality of ablation transducers includes ablation transducers spaced circumferentially around the elongate catheter shaft, ablation transducers axially-spaced along the elongate catheter shaft, or both ablation transducers spaced circumferentially around the elongate catheter shaft and ablation transducers axially-spaced along the elongate catheter shaft.

2. The system of claim 1, wherein at least some of the plurality of ablation transducers are arranged in an array that has an arcuate shape.

3. The system of claim 1, wherein at least some of the plurality of ablation transducers are arranged in an array that has a planar shape.

4. The system of claim 1, wherein the distal end region of the elongate catheter shaft includes a cutout region and wherein at least some of the plurality of ablation transducers are disposed within the cutout region.

5. The system of claim 1, wherein at least some of the plurality of ablation transducers include dual mode transducers.

6. The system of claim 1, wherein at least some of the plurality of ablation transducers include ultrasound transducers.

7. The system of claim 1, wherein at least some of the plurality of imaging transducers include ultrasound transducers.

8. The system of claim 1, wherein at least some of the plurality of ablation transducers are capacitive micromachined ultrasound transducers.

9. The system of claim 1, wherein at least some of the plurality of ablation transducers are dual mode capacitive micromachined ultrasound transducers capable of both an imaging modality and an ablation modality.

10. A system for treating a calcified blood vessel, the system comprising:
an elongate catheter shaft having a distal end region;
a plurality of imaging transducers disposed along the distal end region of the elongate catheter shaft;
a plurality of ablation transducers disposed along the distal end region of the elongate catheter shaft and positioned adjacent to the plurality of imaging transducers;
wherein at least some of the plurality of ablation transducers are capacitive micromachined ultrasound transducers; and
wherein the plurality of ablation transducers includes a plurality of circumferential arrays of ablation transducers spaced circumferentially around the elongate catheter shaft, and/or a plurality of axially-spaced arrays of ablation transducers axially-spaced along the elongate catheter shaft.

11. The system of claim 10, further comprising a plurality of linear fiducial markings disposed between the plurality of imaging transducers and the plurality of ablation transducers.

12. The system of claim 11, wherein the plurality of linear fiducial markings disposed partially along the distal end region of the elongate catheter shaft and circumferentially around the elongate catheter shaft.

13. The system of claim 10, wherein at least some of the plurality of ablation transducers are dual mode transducers.

14. The system of claim 10, wherein at least some of the plurality of ablation transducers are dual mode capacitive micromachined ultrasound transducers capable of both an imaging modality and an ablation modality.

15. The system of claim 10, wherein at least some of the plurality of imaging transducers are capacitive micromachined ultrasound transducers.

16. The system of claim 10, wherein the plurality of ablation transducers operate at power densities ranging from 50 to 80 W/cm².

17. The system of claim 10, wherein the plurality of ablation transducers operate at frequencies ranging 20 Hz to 60 MHz.

18. The system of claim 10, wherein at least some of the circumferential arrays of ablation transducers, at least some of the axially-spaced arrays of ablation transducers, or both have an arcuate shape.

19. The system of claim 10, wherein at least some of the circumferential arrays of ablation transducers, at least some of the axially-spaced arrays of ablation transducers, or both have a planar shape.

20. A method for treating a calcified blood vessel, the method comprising:
advancing a catheter system to a treatment site, the catheter system comprising:
an elongate catheter shaft having a distal end region;
a plurality of imaging transducers disposed along the distal end region of the elongate catheter shaft;
a plurality of ablation transducers disposed along the distal end region of the elongate catheter shaft and positioned adjacent to the plurality of imaging transducers;
wherein the plurality of ablation transducers includes ablation transducers spaced circumferentially around the elongate catheter shaft, ablation transducers axially-spaced along the elongate catheter shaft, or both ablation transducers spaced circumferentially around the elongate catheter shaft and ablation transducers axially-spaced along the elongate catheter shaft; and
activating at least some of the plurality of imaging transducers and/or some of the plurality of ablation transducers.
